(19) United States
(12) Patent Application Publication
(10) Pub. No.: US 2013/0231573 A1
(43) Pub. Date: Sep. 5, 2013

(54) APPARATUS AND METHODS FOR CHARACTERIZATION OF LUNG TISSUE BY RAMAN SPECTROSCOPY

(71) Applicants: Haishan Zeng, Vancouver (CA); Michael Short, Coquitlam (CA); Stephen Lam, Vancouver (CA); Annette McWilliams, Vancouver (CA)

(72) Inventors: Haishan Zeng, Vancouver (CA); Michael Short, Coquitlam (CA); Stephen Lam, Vancouver (CA); Annette McWilliams, Vancouver (CA)

(73) Assignee: BRITISH COLUMBIA CANCER AGENCY BRANCH, Vancouver, BC (CA)

(21) Appl. No.: 13/521,218

(22) PCT Filed: Jan. 21, 2011
(86) PCT No.: PCT/CA11/50040

§ 371 Date: Jul. 9, 2012

Related U.S. Application Data
(60) Provisional application No. 61/297,486, filed on Jan. 22, 2010.
(60) Provisional application No. 61/390,723, filed on Oct. 7, 2010.

(51) Int. Cl. A61B 5/00

(52) U.S. Cl. CPC A61B 5/0075
USPC 600/476

(57) ABSTRACT

Near-infrared Raman spectroscopy can be applied to identify preneoplastic lesions of the bronchial tree. Real-time in vivo Raman spectra of lung tissues may be obtained with a fiber optic catheter passed down the instrument channel of a bronchoscope. Using prototype apparatus, preneoplastic lesions were detected with sensitivity and specificity of 96 and 91% respectively. The use of Raman spectroscopy apparatus and methods in conjunction with other bronchoscopy imaging modalities can substantially reduce the number of false positive results.

### CROSS-REFERENCE TO RELATED APPLICATION

This application claims priority from U.S. patent application No. 61/297,486 entitled ENDOSCOPIC LASER RAMAN SPECTROSCOPY FOR IMPROVING LUNG CANCER DETECTION and filed on 22 Jan. 2010 and 61/390,723 entitled LASER RAMAN SPECTROSCOPY REDUCES FALSE POSITIVE BIOPSIES OF AUTOFLUORESCENCE BRONCHOSCOPY and filed on 7 Oct. 2010. For purposes of the United States, this application claims the benefit under 35 U.S.C. §119 of U.S. patent application No. 61/297,486 filed on 22 Jan. 2010 and No. 61/390,723 filed on 7 Oct. 2010, both of which are hereby incorporated by reference herein.

### TECHNICAL FIELD

The invention relates to the characterization of tissues. The invention may be applied, for example, to provide methods and apparatus for assessing lung tissue for cancer. An example embodiment provides endoscopic apparatus which may be used by a physician to evaluate the likelihood that lesions in lung tissue are cancerous.

### BACKGROUND

Lung cancer is often fatal. The prospects for successful treatment are enhanced by early identification of preneoplastic lesions (lesions that have a high probability of developing into malignant tumours). Preneoplastic lesions of the bronchial tree including moderate and severe dysplasia and carcinoma in situ (CIS) have a high probability of developing into malignant tumours. Localizing these preneoplastic lesions during a bronchoscopy so that further treatment can be administered is key to increasing the patient's chances of survival.

Currently the best method for localizing preneoplastic lesions for further treatment is combined autofluorescence bronchoscopy (AFB) and white light bronchoscopy (WLB). This combination was developed in the 1990s and has made significant improvements to the localization of preneoplastic lesions as described, for example in Lam S, Kennedy T, Unger M, et al. Localization of bronchial intraepithelial neoplastic lesions by fluorescence bronchoscopy. Chest 1998; 113:696-702 and Zellweger M, Grosjean P, Goujon D, Monnier P, van den Bergh H, Wagnieres G. In vivo autofluorescence spectroscopy of human bronchial tissue to optimize the detection and imaging of early cancers. J. Biomed. Opt. 2001; 6:41-51. AFB+WLB has a sensitivity approximately twice that of WLB alone in detecting preneoplasias. However, the average reported specificity of WLB+AFB is only 60% which leads to many false positive identifications as explained, for example, in: Lam S. The Role of Autofluorescence Bronchoscopy in Diagnosis of Early Lung Cancer; in: Hirsch F R, Bunn Jr P A, Kato H, Mulshine J L, eds. IASLC Textbook for Prevention and Detection of Early Lung Cancer. London England; and New York: Taylor & Francis; 2006:149-158; and in Edell E, et al. Detection and Localization of Intraepithelial Neoplasia and Invasive Carcinoma Using Fluorescence-Reflectance Bronchoscopy. Journal of Thorac Oncology. 2009; January; 4(1):49-54.

The suboptimal specificity of WLB+AFB can be partially explained by the fact that selecting which ones of many tissue sites that are typically identified with WLB+AFB to biopsy takes considerable skill and judgment of the bronchoscopist. However, a main reason for the high number of false positives is the low specificity inherent with AFB. Both benign and preneoplastic lesions have similar autofluorescence characteristics. Thus there is still a great need for improved detection methods.

Raman spectroscopy involves directing light at a specimen which inelastically scatters some of the incident light. Inelastic interactions with the specimen can cause the scattered light to have wavelengths that are shifted relative to the wavelength of the incident light (Raman shift). The wavelength spectrum of the scattered light (the Raman spectrum) contains information about the nature of the specimen.

The use of Raman spectroscopy in the study of tissues is described in the following references:
a) Caspers PJ, et al. Roman spectroscopy in biophysics and medical physics. Biophys J 2003; 85:572-580;
b) Huang Z, et al. Rapid near-infrared Raman spectroscopy system for real-time in vivo skin measurements. Opt Lett 2001; 26:1782-1784;
c) Short M A, et al. Development and preliminary results of an endoscopic Raman probe for potential in vivo diagnosis of lung cancers. Opt Lett 2008; 33(7):711-713;
d) Huang Z, et al. Raman spectroscopy of in vivo cutaneous melanin. J of Biomed Opt 2004; 9:1198-1205;
e) Huang Z, et al. Raman Spectroscopy in Combination with Background Near-infrared Autofluorescence Enhances the In Vivo Assessment of Malignant Tissues. Photochem Photobiol 2005; 81:1219-1226;
f) Molckovsky A, et al. Diagnostic potential of near-infrared Raman spectroscopy in the colon: differentiating adenomatous from hyperplastic polyps. Gastrointest Endosc 2003; 57:396-402;
g) Abigail SH, et al. In vivo Margin Assessment during Partial Mastectomy Breast Surgery Using Raman Spectroscopy. Cancer Res 2006; 66:3317-3322;
h) Rajadhyaksha M, et al. In Vivo Confocal Scanning Laser Microscopy of Human Skin II: Advances in Instrumentation and Comparison With Histology. J Invest Dermatol 1999; 113:293-303;
i) Lieber C A, et al. In vivo nonmelanoma skin cancer diagnosis using Raman microspectroscopy. Laser Surg Med 2008; 40(7):461-467;
j) Tu A T. Raman spectroscopy in biology: principles and applications New York, N.Y.: Wiley; 1982;
k) Hanlon EB, et al. Prospects for in vivo Raman spectroscopy Physics in Medicine and Biology 2000; 45:R1-R59;
l) Robichaux-Viehoever A, et al. Characterization of Raman spectra measured in vivo for the detection of cervical dysplasia. Appl. Spectrosc. 2007; 61 pp. 986-997.
m) Guze K, et al. Parameters defining the potential applicability of Raman spectroscopy as a diagnostic tool for oral disease. J. Biomed. Opt. 2009; 14: 0140161-9;
n) Huang Z, et al. Integrated Raman spectroscopy and trimodal wide-field imaging techniques for real-time in vivo tissue Raman measurements at endoscopy. Opt. Lett. 2009; 34:758-760;
o) Huang Z, et al. Near-infrared Raman spectroscopy for optical diagnosis of lung cancer. Int. J. Cancer 2003; 107:1047-1052;
p) Magee N D, et al. Ex Vivo diagnosis of lung cancer using a Raman miniprobe. Journal of Physical Chemistry B 2009; 113:8137-8141;
q) Short M A, et al. Development and preliminary results of an endoscopy Raman probe for potential in-vivo diagnosis of lung cancers. Optics Letters 2008; 33(7):711-713;
r) Shim MG, et al. Study of fiber optic probes for in vivo medical Raman spectroscopy. Applied Spectroscopy 1999; 53: 619-627;
s) Yamazaki H, et al. The diagnoses of lung cancer using 1064 nm excited near-infrared multichannel Raman spectroscopy. Radiation Medicine 2003; 21:1-6;
t) Nazemi JH, et al. Lipid concentrations in human coronary artery determined with high wavenumber Raman shifted light. J. Biomed. Opt. 2007; 14(3):0340091-6;
u) Koljenovi´c S, et al. Raman nzicrospectroscopic mapping studies of human bronchial tissue. J. Biomed. Opt. 2004; 9:1187-1197;
v) Movasaghi Z, et al. Raman spectroscopy of biological tissues. Applied Spectroscopy Reviews 2007; 42:493-541; and
w) Percot, A. et al. Direct observation of domains in model stratum corneum lipid mixtures by Raman spectroscopy. Biophysical Journal 2001; 81:2144-2153.
   All of these references are hereby incorporated herein by reference.

The use of optical apparatus which applies Raman spectroscopy to analyze light collected using confocal techniques is described in:
x) Caspers PJ, et al. Automated depth-scanning confocal Raman microspectrometer for rapid in vivo determination of water concentration profiles in human skin. J Raman Spectrosc 2000; 31:813-818;
y) Caspers PJ, et al. In vivo confocal Roman microspectroscopy of the skin: noninvasive determination of molecular concentration profiles. J Invest Dermatol 2001; 116:434-442;
z) Caspers PJ, et al. Monitoring the penetration enhancer dimethyl sulfoxide in human stratum corneum in vivo by confocal Raman spectroscopy. Pharm Res 2002; 19:1577-1580.

All of these references are hereby incorporated herein by reference.

A sensitive, specific non-invasive tool for characterizing suspicious lesions and other tissues would provide a valuable alternative to the use of biopsies and histopathologic examination of the extracted tissues.

### SUMMARY OF THE INVENTION

This invention has a number of aspects. These aspects include: apparatus useful for assessing the pathology of lung tissue in vivo; methods useful for assessing the pathology of lung tissue in vivo; apparatus for processing tissue Raman spectroscopy data and generating a measure of the likelihood that the spectra correspond to cancerous or pre-cancerous tissues; methods for processing tissue Raman spectroscopy data and generating a measure of the likelihood that the spectra correspond to cancerous or pre-cancerous tissues; non-transitory media containing computer-readable instructions that, when executed by a data processor cause the data processor to execute a method for processing tissue Raman spectroscopy data and generating a measure of the likelihood that the spectra correspond to cancerous or precancerous tissues.

One aspect of the invention provides methods and apparatus useful for the non-invasive analysis of lung tissue for the diagnosis of disease or physiological states by detection and measurement of the Raman spectra.

Some embodiments of the invention provide methods and apparatus for acquiring and analyzing point Raman spectra to provide objective measures for evaluating tissues, for example, tissues at candidate locations in the lungs or bronchial tree. Some embodiments provide fast and objective measures of whether a lesion is preneoplastic, malignant or neither.

In some embodiments the method and apparatus are adapted to distinguish between the group consisting of the classes of Normal, Inflamed, Hyperplasia, Mild Dysplasia, and the group consisting of the classes of Moderate Dysplasia, Severe Dysplasia, Carcinoma in Situ (CIS) and Tumor. The first 4 classes are considered benign and the last 4 malignant.

One aspect of the invention provides an apparatus for tissue characterization comprising a Raman spectrometer configured to generate a Raman spectrum, a Raman spectrum analysis unit configured to measure at least one characteristic of the Raman spectrum, and a feedback device driven in response to the measured characteristic. The at least one characteristic including one or more spectral features within a relative wavenumber range from 1500±10 cm⁻¹ to 3400±10 cm⁻¹.

In some embodiments apparatus is further configured to process Raman spectra to provide smoothed 2^{nd} order derivative spectra. This may be achieved, for example, by applying a Savitzky-Golay six point quadratic polynomial. Tissues may be characterized on the basis of features in the smoothed 2^{nd} order derivative spectra.

In some embodiments the apparatus is configured to characterize the tissues by: characterizing the tissue in a first category if a posterior probability of a characteristic of the tissue is less than a first threshold; characterizing the tissue in a second category if the posterior probability of the characteristic of the tissue is greater than a second threshold; and characterizing the tissue in a third category if the posterior probability of the characteristic of the tissue is between the first and second thresholds. In some embodiments the first threshold represents a cutoff of 0.3±10% and the second threshold represents a cutoff of 0.7±10%. For example, the first threshold may be a cutoff of 0.3 and the second threshold may be a cutoff of 0.7.

Another aspect of the invention provides a method for tissue characterization involving receiving at least one Raman spectrum of a lung tissue, measuring at least one characteristic of the Raman spectrum, characterizing the tissue in response to the measured characteristic, and generating an indication of the characterization of the tissue. Characterizing the tissue is based at least in part on one or more features of the Raman spectrum in the relative wavenumber range of 1500±10 cm⁻¹ to 3400±10 cm⁻¹.

In some embodiments, a smoothed 2nd order derivative spectrum is calculated. This may be done, for example, by applying a Savitzky-Golay six point quadratic polynomial to each Raman spectrum.

In some embodiments characterizing the tissues comprises: characterizing the tissue in a first category if a posterior probability of a characteristic of the tissue is less than a first threshold; characterizing the tissue in a second category if the posterior probability of the characteristic of the tissue is greater than a second threshold; and characterizing the tissue in a third category if the posterior probability of the characteristic of the tissue is between the first and second thresholds. In some embodiments the first threshold represents a cutoff of 0.3±10% and the second threshold represents a cutoff of 0.7±10%. For example, the first threshold may be a cutoff of 0.3 and the second threshold may be a cutoff of 0.7.

Another aspect of the invention provides a non-transitory tangible computer-readable medium storing instructions for execution by at least one data-processor that, when executed by the data-processor cause the data processor to execute a method for characterizing tissue comprising the steps of processing at least one Raman spectrum of a lung tissue, characterizing the lung tissue in response to the Raman spectrum and generating an indication of the characterization of the lung tissue. Characterizing the tissue is based at least in part on one or more features of the Raman spectrum in the relative wavenumber range of 1500±10 cm⁻¹ to 3400±10 cm⁻¹.

Additional aspects of the invention and features of example embodiments of the invention are described in the following description and/or illustrated in the accompanying drawings.

### BRIEF DESCRIPTION OF THE DRAWINGS

The accompanying drawings illustrate non-limiting embodiments of the invention.

FIG. 1 is a block diagram of a diagnostic apparatus according to an example embodiment of the invention.

FIG. 2 is a block diagram of an apparatus according to another example embodiment of the invention.

FIG. 2A is a photograph of a prototype diagnostic apparatus.

FIG. 3A is a graph of a raw Raman spectrum.

FIG. 3B is a graph of the Raman spectrum of FIG. 3A with a polynomial curve fit to the fluorescence background.

FIG. 3C is a graph of the Raman spectrum of FIG. 3A with the fluorescence background subtracted.

FIG. 4A is a photograph of a lesion under white light.

FIG. 4B is a blue light excited fluorescence photograph of the same lesion.

FIG. 4C is a blue light and Raman spectrometer excited fluorescence photograph of a suspected lesion.

FIG. 5A is a graph of example average Raman spectra from a dataset.

FIG. 5B is a graph of another example average Raman spectra from a dataset.

FIG. 5C is a graph of a further example average Raman spectra from a dataset.

FIG. 5D is a graph of example Raman spectra for various classifications of lesions.

FIG. 6 is a graph of an example posterior probability plot of predicted and known pathology.

FIG. 7 is a graph of example receiver operator characteristics of example Raman spectra.

FIG. 8 is a graph showing example Raman spectra for various reference materials.

### DESCRIPTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive, sense.

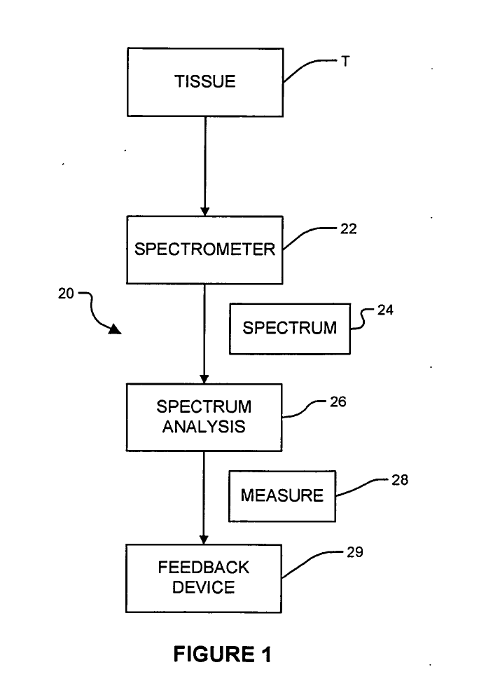

FIG. 1 is a block diagram of apparatus 20 according to an example embodiment of the invention. Apparatus 20 comprises a Raman spectrometer 22 which is configured to determine a Raman spectrum 24 for a small volume of a tissue T. Tissue T may be lung tissue.

Obtaining in vivo Raman spectra of lung tissue can be complicated by the problems that a fiber optic or other flexible probe is generally required to carry light to a spectrometer from the lung tissue and this can result in reduced efficiency of light collection. Another problem is that frequent and uncontrollable lung movements make it difficult to maintain focus on a particular area of tissue for more than a few seconds. These issues can be addressed by using components to reduce fiber emission as described in Shim MG, et al. Study of fiber optic probes for in vivo medical Raman spectroscopy. Applied Spectroscopy 1999; 53: 619-627 and taking steps to promote a high signal to noise ratio as described in Huang Z et al. Rapid near-infrared Raman spectroscopy system for real-time in vivo skin measurements. Optical Letters 2001; 26:1782-1784. Short M A, et al. Development and preliminary results of an endoscopy Raman probe for potential in-vivo diagnosis of lung cancers. Optics Letters 2008; 33(7):711-713 describes a prototype Raman spectroscopy system suitable for acquiring Raman spectra from lung tissues.

A spectrum analysis component 26 receives Raman spectrum 24 and processes the Raman spectrum to obtain a measure 28 indicative of the pathology of the tissue for which Raman spectrum 24 was obtained. Measure 28 controls a feedback device 29. Feedback device 29 may, for example, comprise a lamp, graphical indication, sound, display or other device which provides a human-perceptible signal in response to measure 28.

Measure 28 is based at least in part upon features of the Raman spectrum found in the wavenumber range of 1500 cm⁻¹ to 3400 cm⁻¹.

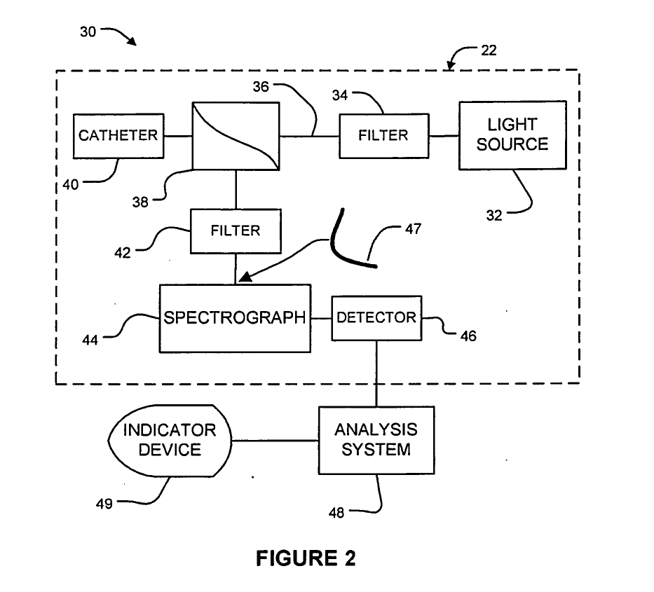

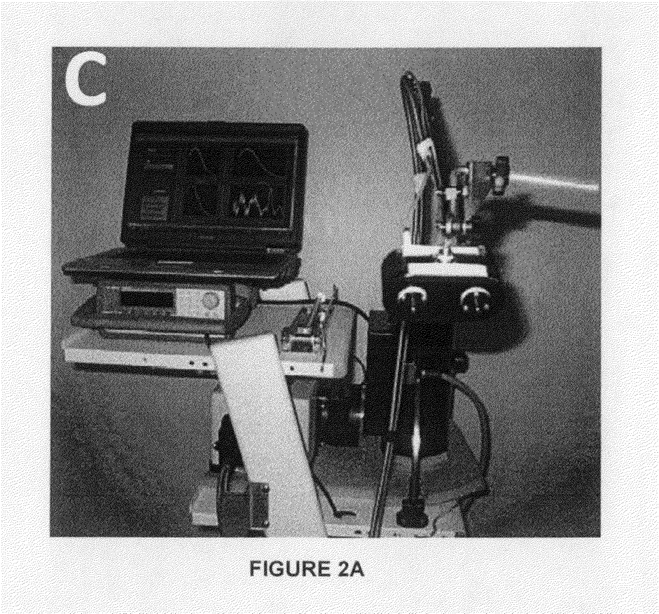

FIG. 2 is a block diagram of apparatus 30 according to another example embodiment of the invention. In FIG. 2, Raman spectrometer 22 is shown to comprise a light source 32. Light source 32 is a monochromatic light source and may, for example, comprise a laser. Light source 32 may, for example, comprise an infrared laser. In an example embodiment, the laser generates light having a wavelength of 785 nm.

It is desirable to avoid exposing tissues to excessive amounts of radiation. This may be achieved by appropriate selection of light source 32, control of the light source, and/or providing attenuation downstream from the light source.

Light from light source 32 is filtered by filter 34 and coupled into optical fiber 36. The light passes through a beamsplitter 38 into a catheter 40. Catheter 40 may, for example, extend down the instrument channel of a bronchoscope. In an example embodiment, catheter 40 has a diameter of 1.8 mm so that it can fit through the 2.2 mm diameter instrument bore of a bronchoscope. Light that emerges from the distal end of the catheter 40 illuminates tissues adjacent the end of catheter 40 where some of the light undergoes Raman scattering. Some of the Raman scattered light enters catheter 40 and is carried to spectrograph 44 by way of beamsplitter 38 and filter 42.

Spectrograph 44 and detector 46 work together to produce a Raman spectrum of the light incident at spectrograph 44. Information characterizing the Raman spectrum is passed to an analysis system 48. Preferably Raman spectra are acquired within a short data acquisition time such as 1 second.

Spectrum analysis system 48 may comprise a programmed data processor such as a personal computer, an embedded computer, a microprocessor, a graphics processor, a digital signal processor or the like executing software and/or firmware instructions that cause the processor to extract the specific spectral characteristics from the Raman spectra. In alternative embodiments spectrum analysis system 48 comprises electronic circuits, logic pipelines or other hardware that is configured to extract the specific spectral characteristics or a programmed data processor in combination with hardware that performs one or more steps in the extraction of the specific spectral characteristics.

It is convenient but not mandatory for spectrum analysis system 48 to operate in real time or near real time such that analysis of a Raman spectrum is completed at essentially the same time or at least within a few seconds of the Raman spectrum being acquired.

In FIG. 2, 47 indicates a 50 μm diameter fiber used to calibrate the spectrometer.

Spectrum analysis system 48 is connected to control an indicator device 49 according to a measure derived from the specific spectral characteristics extracted from the Raman spectrum by spectrum analysis system 48.

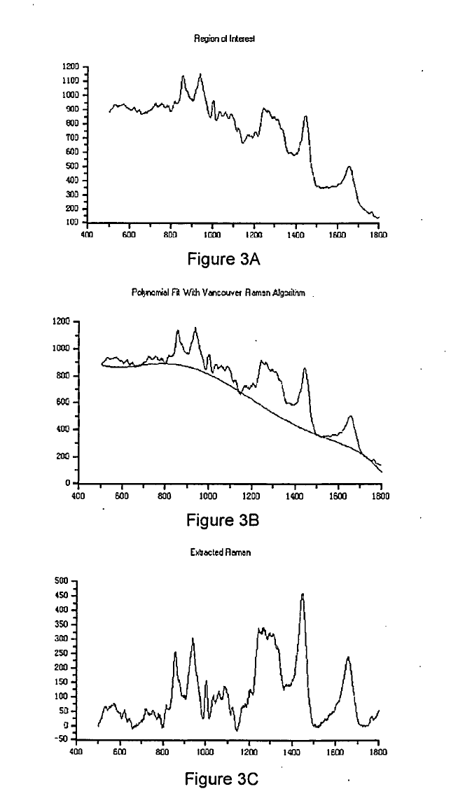

The measured Raman spectra are typically superimposed on a fluorescence background, which varies with each measurement. It is convenient for spectrum analysis system 48 to process received Raman spectra to remove the fluorescence background and also to normalize the spectra. Removal of fluorescence background may be achieved, for example using the Vancouver Raman Algorithm as described in Zhao J, et al. Automated Autofluorescence Background Subtraction Algorithm for Biomedical Raman Spectroscopy. Appl. Spectrosc. 2007; 61:1225-1232, which is hereby incorporated herein by reference. The Vancouver Raman Algorithm is an iterative modified polynomial curve fitting fluorescence removal method that takes noise into account. FIGS. 3A, 3B and 3C respectively show a raw Raman spectrum, the Raman spectrum of FIG. 3A with a polynomial curve fit to the fluorescence background and the Raman spectrum of FIG. 3A with the fluorescence background as modeled by the polynomial curve subtracted.

Normalization may be performed, for example, to the area under curve (AUC) of each spectrum. For example, each spectrum may be multiplied by a value selected to make the AUC equal to a standard value. For convenience in displaying the spectra, the normalized intensities may be divided by the number of data points in each spectrum.

Spectrograph 44 and spectrum analysis system 48 are configured to obtain and analyze Raman spectra that include at least part of the 1500 cm⁻¹ to 3400 cm⁻¹ range. The inventors have determined that this range provides particular advantages as it avoids the very strong lung tissue autofluorescence found in the 0 to 2000 cm⁻¹ range and yet still contains significant biomolecular information that is useful for tissue characterization.

Spectrum analysis system 48 may apply multivariate data analysis to classify tissues according to their Raman spectra in the 1500 cm⁻¹ to 3400 cm⁻¹ range. For example, a particular spectrum may be analyzed by performing a principle component analysis (PCA). PCA may be performed on part or all of the range of the acquired Raman spectra.

PCA involves generating a set of principle components which represent a given proportion of the variance in a set of training spectra. For example, each spectrum may be represented as a linear combination of a set of a few PCA variables. The PCA variables may be selected so that they account for at least a threshold amount (e.g. at least 70%) of the total variance of a set of training spectra.

Principal components (PCs) may be derived by performing PCA on a standardized spectral data matrix to generate PCs. The PCs generally provide a reduced number of orthogonal variables that account for most of the total variance in original spectra.

PCs may be used to assess a new Raman spectrum by computing a variable called the PC score, which represents the weight(s) of particular PC(s) in the Raman spectrum being analyzed.

Linear discriminant analysis (LDA) can then be used to derive a function of the PC scores (a discriminate function) which indicates whether or not the tissue is normal.

The discriminate function may subsequently be applied to categorize an unknown tissue based on where a point corresponding to the PC scores for a Raman spectrum of the unknown tissue is relative to the discriminate function line.

Spectrum analysis system 48 may be configured to perform linear discriminant analysis and/or principal component analysis on the Raman spectra in the 1500 cm⁻¹ to 3400 cm⁻¹ range to discriminate between healthy and unhealthy lung tissue. An example of this is provided below.

FIG. 2A is a photograph showing apparatus according to a prototype embodiment. The apparatus is mounted on a cart so that it can be brought close to a patient.

One application of apparatus 20 or 30 is to characterize lesions that have been identified as being of interest using a different modality, for example, WLB and AFB. It is convenient for catheter 40 to be carried by the same instrument (e.g. a bronchoscope) used to identify the lesions of interest. This facilitates the use of Raman spectroscopy to characterize a lesion immediately upon the lesion being observed. A physician can use the bronchoscope to identify lesions of interest by viewing lung tissue under one or more appropriate imaging modes. When a lesion of interest has been located the physician may trigger the acquisition and analysis of a Raman spectrum of the lesion of interest without moving the bronchoscope. This may be done, for example, by pressing a button or using another user interface modality to command the apparatus to acquire a Raman spectrum. In some embodiments, the physician immediately receives the results of an automated analysis of the Raman spectrum. Based on the results of the automated analysis the physician can decide on further actions such as whether or not to take a biopsy of the lesion of interest.

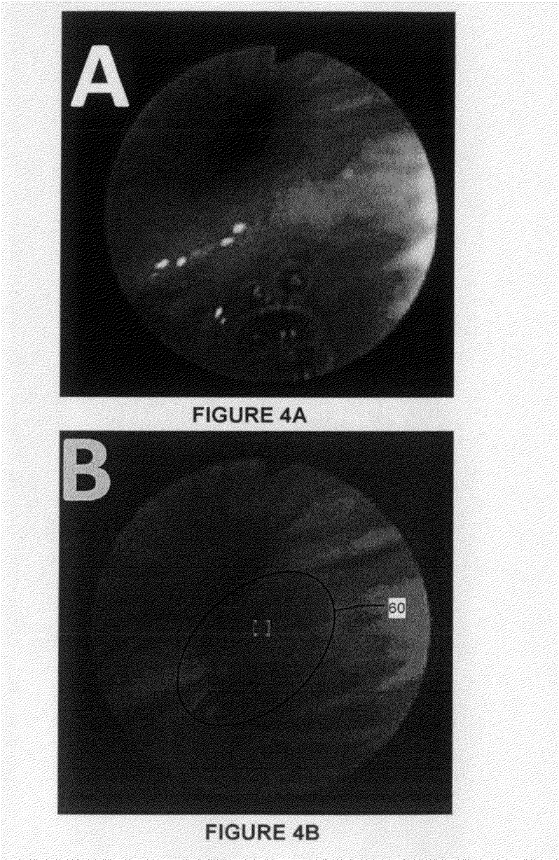

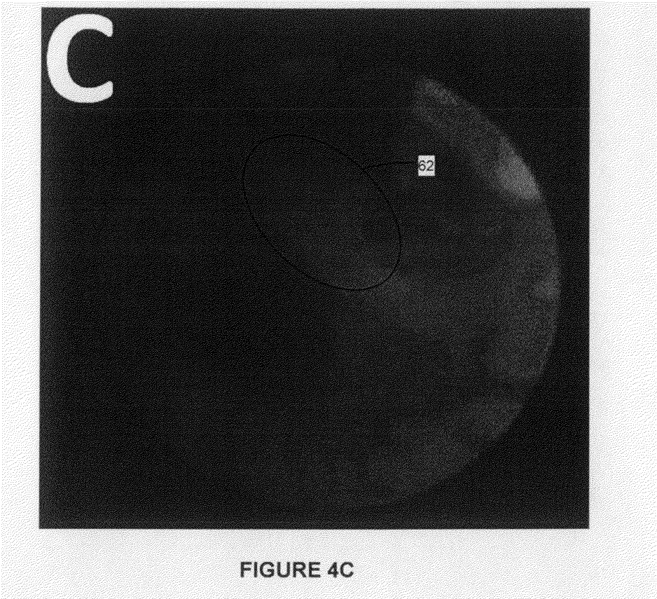

FIG. 4A is a photograph showing a lesion imaged under white light and FIG. 4B is a photograph of the same location shown in FIG. 4A viewed as a blue light excited fluorescence image. FIG. 4B was obtained using an Onco-LIFE™ fluorescence endoscopy system from Xillix Technologies Corp. of Vancouver, Canada. In FIG. 4B, green represents normal tissue, and dark red (in area 60 for example) represents diseased tissue.

FIG. 4C is a photograph showing another suspected lesion being excited simultaneously with blue light to generate a fluorescence image and with 785 nm light from a catheter 40 of a Raman spectrometer, wherein an area generally indicated by area 62 is red and the remaining area is predominately green.

The invention is further described with reference to the following specific example, which is not meant to limit the invention, but rather to further illustrate it.

### Example 1

A near-infrared Raman system of the type illustrated in FIG. 2 was used to collect real-time, in vivo lung spectra of lesions in lung tissues. The lung tissues were from 26 people selected from a group of 46 people undergoing bronchoscopy. A bronchoscopist identified lesions to biopsy using combined WLB and AFB. Of the 46 participants, 26 were found to have lesions that the bronchoscopist elected to biopsy. Raman spectra were obtained from these lesions using apparatus as described herein. 129 Raman spectra were measured. Clear in vivo Raman spectra were obtained in one second exposures.

Biopsies were taken of the same locations, and classified by a pathologist. Eight classifications were used according to World Health Organization criteria (see, for example, Travis WD, et al. Histologic and graphical text slides for the histological typing of lung and pleural tumors. In: World Health Organization Pathology Panel: World Health Organization International Histological Classification of Tumors, 3rd ed. Berlin: Springer Verlag; 1999, p. 5). These eight classifications were: Normal epithelium; Hyperplasia (including goblet cell hyperplasia and basal/reserve cell hyperplasia); Metaplasia (including immature squamous metaplasia and squamous metaplasia); Mild Dysplasia; Moderate Dysplasia; Severe Dysplasia: CIS: and Invasive squamous cell Carcinoma (IC). The presence or absence of inflammatory changes was also recorded. In the following discussion, ≧MOD means lesions with pathology of moderate dysplasia or worse and ≦MILD means lesions with pathology of mild dysplasia or better.

Of the 129 Raman spectra that were obtained, 51 were from sites with pathologies of ≧MOD, the rest were from sites with pathologies of mild dysplasia or better (≦MILD).

An ambient background signal was subtracted from the raw data of each spectrum, before calibrating for the sensitivity of the system as a function of wavelength. The pre-processed spectra were each processed in three different ways.

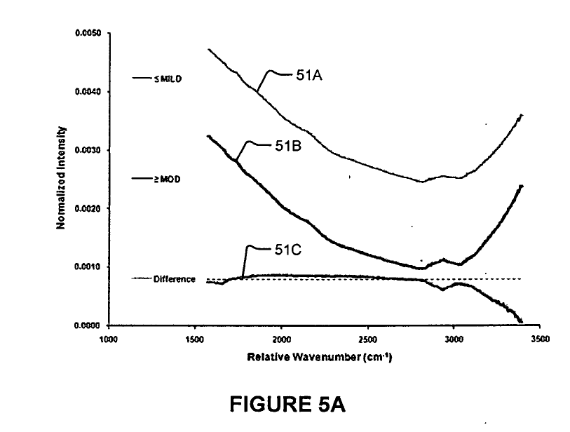

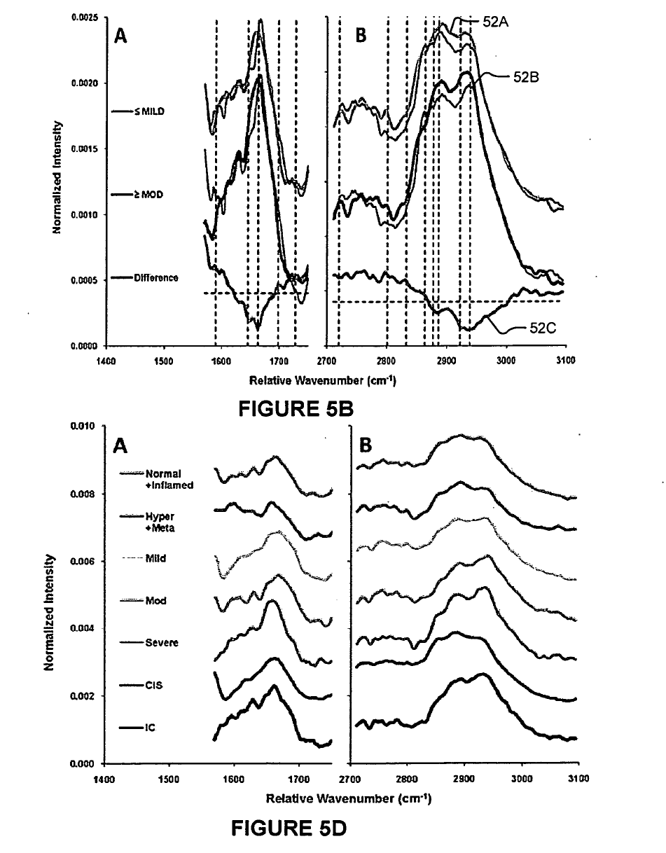

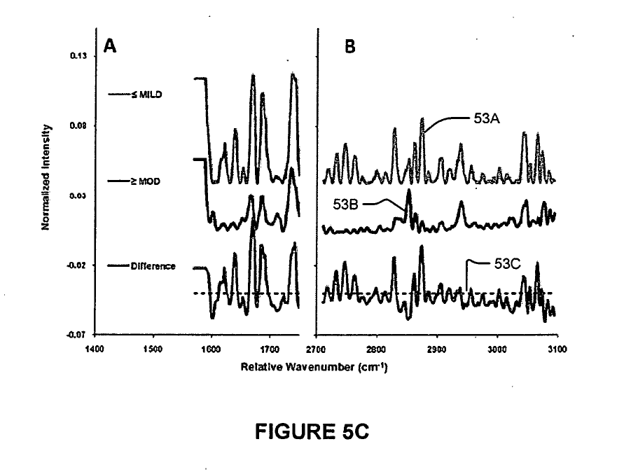

A first dataset (dataset A) was obtained by performing a 3-point smoothing operation on each pre-processed spectrum and normalizing for intensity variations by summing the area under each curve and dividing each variable in the smoothed spectrum by this sum. FIG. 5A shows average spectra for the data from dataset A from sites with pathology ≦MILD (curve 51A) and ≧MOD (curve 51B). Curves 51A and 51B are shifted on the intensity scale for clarity. Curve 51C shows the result of subtracting the average ≦MILD spectra from the average ≧MOD spectra (not on the same intensity scale). The horizontal dashed line is at zero intensity.

Inspection of FIG. 5A shows a substantial autofluorescence contribution to the dataset A spectra with relatively small Raman peaks around 1600, 2150, and 2900 cm⁻¹. A low intensity broad peak centered at 2150 cm⁻¹ and the intense emission rising above 3100 cm⁻¹ are assigned to water molecule vibrations.

A second dataset (dataset B) was obtained by performing a 3-point smoothing operation and then subtracting autofluorescence by a modified polynomial fitting routine as described in Zhao J, et al. Automated autofluorescence background subtraction algorithm for biomedical Raman spectroscopy. Applied Spectroscopy 2007; 61:1225-1232. The resulting spectra were normalized as described for the first dataset.

FIG. 5B shows the average Raman spectra from dataset B of ≦MILD lesions (curve 52A) and ≧MOD lesions (curve 52B). Curve 52C shows the result of subtracting the average ≦MILD spectra from the average ≧MOD spectra (not on the same intensity scale). Curves 52A and 52B show significant differences as determined by a t-test statistic (p. 0.05) at 13 wavenumber locations (indicated by vertical dashed lines in the Figure). These locations either correspond to peaks or shoulders in the spectra. Two ranges (A and B) are shown as clear Raman peaks were not observed outside of these ranges. An approximate fit to each average spectrum was obtained with a least squares weighted sum of all the references measured. These fits are indicated by solid black lines and show relative increases in DNA, hemoglobin, phenylalanine, and triolein for ≧MOD lesions, and a corresponding drop in collagen.

FIG. 5D shows in vivo Raman spectra processed as dataset B for lesions of various classifications. Two wavenumber ranges (A and B) are shown as clear Raman peaks were not observed outside of these ranges. The spectra in range A were, on average, 5 times less intense than those in range B.

The broad peaks near 1663 cm⁻¹ probably correspond to a combination of ν (C═O) amide I vibrations and ν2 water molecule bending motions. The broad peak around 2900 cm⁻¹ is assigned to a combination of lipid (C—H) peaks (2833+2886 cm¹) and generic protein vibrations at 2938 cm⁻¹.

FIG. 5D shows other small peaks or shoulders at: 1589, 1646, 1698, 1727, 2720, 2801, 2863, 2877 and 2921 cm⁻¹ that appear to correspond with peaks of various amino acids, lipids, and proteins. Between 1750 and 2700 cm⁻¹ (a range not shown in FIG. 5D) there were a number of narrow peaks with very low intensities, apart from the broad emission at 2150 cm⁻¹, that did not seem to vary for different lung sites. This spectral region is not noted for any significant Raman peaks although there are reports of some weak Raman emissions mainly due to carbon and nitrogen modes that were in approximate agreement with some of the very low intensity peaks observed.

A third dataset, (dataset C) was prepared by applying a Savitzky-Golay six point quadratic polynomial to each pre-processed spectrum to calculate a smoothed 2nd order derivative spectrum. This technique is described for example, in Savitzky A, et al. Smoothing and differentiation of data by simplified least squares procedure Analytical Chemistry 1964; 36:627-1639. Summing the squared derivative values of a spectrum and then dividing each variable by this sum was used for normalization.

The second derivative spectra of dataset C, over the ranges where significant differences (p. 0.05) between different pathology groups were apparent, is shown in FIG. 5C. Curve 53A is average processed data from sites with pathology Curve 53B is average processed data from sites with pathology ≧MOD. Curves 53A and 53B have been shifted on the intensity scale for clarity. Curve 53C shows the result of subtracting the average ≦MILD spectra from the average ≧MOD spectra (not on the same intensity scale). The horizontal dashed line is at zero intensity. Two wavenumber ranges ((A) 1550-1800 cm⁻¹ and (B) 2700-3100 cm⁻¹) are shown. Clear Raman peaks were only observed in these ranges.

Datasets A, B, and C were analyzed separately using statistical software (Statistica™ 6.0, from StatSoft Inc. of Tulsa, Okla. USA). Principle components (PCs) for all the spectra in each dataset were computed to reduce the number of variables. Student's t-tests were used on PCs that accounted for 0.1% or more of the variance to determine those most significant at separating spectra into two pathology groups: ≧MOD and ≦MILD. A linear discrimination analysis (LDA) with leave-one-out cross validation was used on the most significant PCs. To avoid over fitting the data, the number of PCs used in the LDA were limited to one third (17) of the total number of cases of the smallest subgroup, i.e. 51 ≧MOD spectra.

Leave-one-out cross validation procedures may be used in order to prevent over training. Leave-one-out cross validation involves removing one spectrum from the data set and repeating the entire algorithm, including PCA and LDA, using the remaining set of spectra. The resulting optimized algorithm is then used to classify the withheld spectrum. This process may be repeated until each spectrum has been individually classified.

The complete analyses of spectra from datasets A, B and C were redone a second time as described above except that all the spectra with IC pathology (24) were dropped from each dataset. 27 spectra in each dataset remained with ≧MOD pathology classification, and thus only 9 PCs were used in the LDA cross validation model.

Statistical analysis on spectra from datasets A, B and C led to significantly different results as can be seen from Table I. Spectra from dataset A were the worst in predicting the pathology ≧MOD with 80% sensitivity and 72% specificity. Removing the IC spectra from analyses resulted in a substantially worse sensitivity with only a modest increase in specificity. If spectra were only classified when the posterior probability was ≧0.7 or ≦0.3, then 80% sensitivity and 77% specificity were obtained at the cost of only being able to classify 99 out of 129 spectra (77%).

TABLE I
Classification Results

| Dataset | Sub Dataset | Sensitivity | Specificity | # of Spectra Classified |
|---|---|---|---|---|
| A | All Data | 80% | 72% | 51 ≧ MOD, 78 ≦ MILD |
| | All Data-IC | 55% | 76% | 27 ≧ MOD, 78 ≦ MILD |
| | 70, 30 | 80% | 77% | 35 ≧ MOD, 64 ≦ MILD |
| B | All Data | 80% | 79% | 51 ≧ MOD, 78 ≦ MILD |
| | All Data-IC | 89% | 79% | 27 ≧ MOD, 78 ≦ MILD |
| | 70, 30 | 83% | 84% | 40 ≧ MOD, 63 ≦ MILD |
| C | All Data | 90% | 91% | 51 ≧ MOD, 78 ≦ MILD |
| | All Data-IC | 96% | 91% | 27 ≧ MOD, 78 ≦ MILD |
| | 70, 30 | 93% | 96% | 45 ≧ MOD, 68 ≦ MILD |

Analysis of dataset B spectra showed an improvement in pathology prediction compared to dataset A spectra with 80% sensitivity and 79% specificity. Removing the IC spectra from the analyses resulted in a substantially better specificity (89%) with the sensitivity unchanged. When using cut-off lines at 0.7 and 0.3, the sensitivity and specificity were 83 and 84% respectively, and 80% of the 129 spectra were classified.

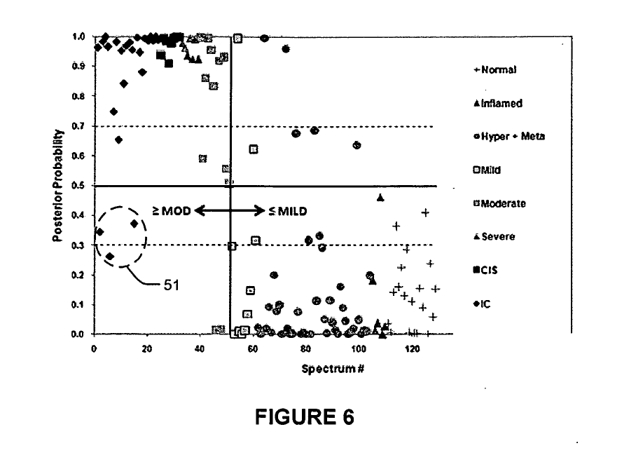

The best result was obtained by analyzing spectra processed with the second order derivative (dataset C). FIG. 6 is a posterior probability plot of predicted pathology compared to known pathology. Statistical analysis of dataset C was performed using a leave-one-out cross-validation. 17 PCA components were used in the LDA model. 90% sensitivity and 91% specificity were obtained using all the spectra. In this case only three IC spectra 51 were mis-classified (see FIG. 6). Dropping all the IC spectra from analyses resulted in the sensitivity increasing to 96% with the specificity unchanged at 91%, and when using the 0.7 and 0.3 cut off lines both sensitivity and specificity increased with 88% of spectra classified.

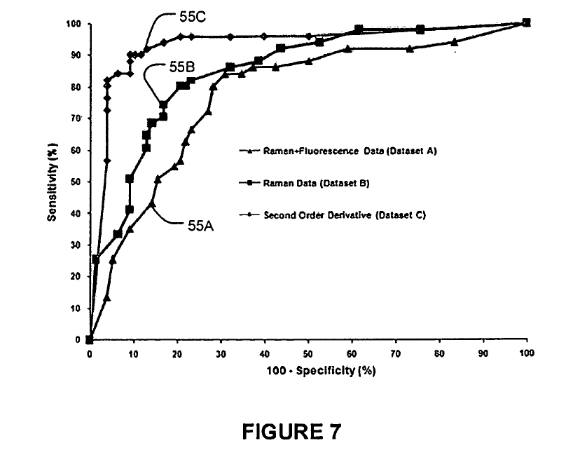

The receiver operator characteristics (ROC) for all the three datasets are shown in FIG. 7. FIG. 7 shows how the sensitivity and specificity change when moving the cut line from 0 to 100% in the LDA posterior probability plots. Dataset A corresponds to curve 55A. Dataset B corresponds to curve 55B. Dataset C corresponds to curve 55C. One can clearly see the superiority of second order derivative processed spectra (dataset C). The fractional areas under each ROC curve were 0.78, 0.85 and 0.92 for spectra analyzed in datasets A, B and C respectively.

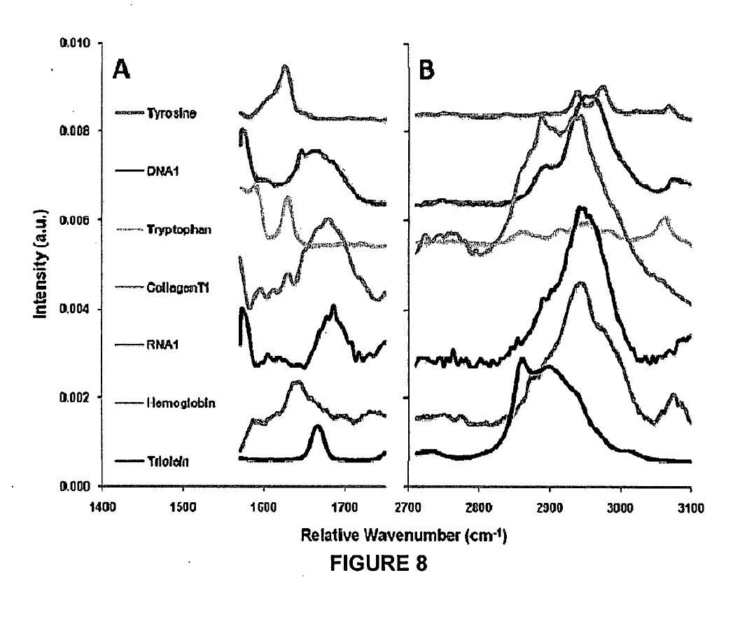

Raman spectra of reference materials that are the main contributors to emissions from human epithelia and connective tissues were obtained for comparison. These were: DNA purified from a human placenta, RNA from baker's yeast, phenylalanine, tyrosine, tryptophan, triolein (an abundant lipid of the bronchial mucus), collagen from human lung, and human hemoglobin. Most reference samples were obtained from Sigma-Aldrich Canada Ltd with reference #'s DNA (D4642), RNA (R6750), phenylalanine (P2126), tyrosine (T3754), tryptophan (T0254), triolein (T7140), and human lung collagen (CH783). The hemoglobin was from the blood sample of a volunteer. The references were used neat in their supplied state without further processing. Spectra were obtained using the same equipment as the in vivo measurements by supporting the Raman catheter a few millimetres above each sample. The data were pre-processed in the same way as the in vivo data and then further processed as for dataset B spectra. FIG. 8 shows the Raman spectra for the reference materials. The spectra have been shifted along the intensity axis for clarity. The spectra have features consistent with those reported in the literature.

Although the relative wavenumber range of 1500 cm⁻¹ to 3400 cm⁻¹ is not free of autofluorescence, it was found that autofluorescence was an order of magnitude less than found over the usual range of 0 to 2000 cm⁻¹. Furthermore, despite there being fewer Raman peaks in the measurement range and although there did not appear to be any consistent trend in peak changes (See e.g. FIG. 5D) as the pathology of the tissue goes through the various changes from normal to IC, there were statistically significant differences in the spectra for sites with different pathologies.

The statistical analysis of dataset A spectra may be explained by the fact that the site selection process was biased toward selecting only sites that were identified by AFB imaging. However, it is known that this results in a less than optimal specificity. Since it is generally not difficult to identify IC using a combination of WLB and AFB, dropping the IC spectra from the data analyses may improve detection of early stage disease. In the case of dataset A spectra this reasoning proved false with only 55% of spectra from ≧MOD sites identified. The obvious explanation for this is that autofluorescence dominates the spectra, and that this autofluorescence is similar for all sites measured except those with IC.

The use of cut-off lines in analyses can be beneficial when it is not possible to consistently get good quality spectra. Patient involuntary movements may be one cause of this problem. Significant mucus or water on the tissue surface may be another cause.

In some embodiments, analysis system 48 is configured to determine whether or not an obtained spectrum satisfies a statistical standard of being ≧MOD or ≦MILD and to signal to a user if this statistical standard is not met. Since the apparatus is intended to be used in clinical settings and to produce results essentially in real time, such embodiments enable the bronchoscopist to immediately take another spectrum if the previous spectrum did not meet the statistical standard (e.g. pass the cutoff). Any sites that failed after several attempts could be biopsied. The cut-off lines should not be made too strict otherwise because this would defeat the object of decreasing the number of false positives. In the study on which this work is based, 0.7 and 0.3 posterior probability cut-offs were chosen.

Analyses of dataset B spectra produced better results than dataset A, although there were some spectra from IC sites that were mis-classified. The reason for this was most likely sampling errors, as IC lesions may contain areas other than the histologically malignant epithelium (i.e desmoplatic stroma). Sampling of the adjacent reactive or inflamed non-neoplastic tissue is another possibility for the mis-classified samples. Removing the IC spectra from analyses did increase the specificity by 9% with the sensitivity unchanged (see Table I). Posterior probability cut off lines made modest improvements to the sensitivity and specificity.

The second order derivative spectra (dataset C) were the best at separating ≧MOD and ≦MILD tissue with 90% sensitivity and 91% specificity. Dropping the IC spectra sees the sensitivity rise by 6% with no loss in specificity. Apart from the IC spectra, the other mis-classified sites were those with moderate dysplasia, mild dysplasia, metaplasia, and hyperplasia pathologies. Sampling errors may again explain these mis-classifications. An alternative explanation for the mis-classifications is that the Raman spectra contain biomolecular information, with no obvious histological counterpart on whether a lesion will develop into late stage disease or not.

It is not fully understood why dataset C produced improved sensitivity and specificity values. While the inventors do not wish to be bound by any particular theory, one reason may be that inaccuracies in the polynomial fitting of the substantial autofluorescence introduce an uncorrelated variance into dataset B.

The methods described above may be varied in various ways. For example other techniques for removing background fluorescence may be applied. Shifted subtracted Raman spectroscopy as described in Magee N D, et al. Ex Vivo diagnosis of lung cancer using a Raman miniprobe. Journal of Physical Chemistry B 2009; 113:8137-8141 may be applied. Known techniques for removing background fluorescence all have advantages and disadvantages as explained by Zhao et al. Automated autofluorescence background subtraction algorithm for biomedical Raman spectroscopy. Applied Spectroscopy 2007; 61:1225-1232. The method described by Zhao et al. tends to work well for non-complex background fluorescence and is fast for real time clinical applications. Generating a derivative spectrum is also a fast process that can be done in the clinic in real-time, this combination may be the optimal choice with intensely fluorescing tissue.

In conclusion it appears that point Raman spectroscopy as described herein can be applied to significantly reduce the number of false positive biopsies while only marginally reducing the sensitivity of WLB and AFB to the detection of preneoplastic lung lesions. Although it may be considered better to have a 40% false positive rate than incur any loss in detection sensitivity, the slight loss incurred with the adjunct use of Raman spectroscopy may not be realized in practice. First, bronchoscopists currently have to make partially subjective decisions when using WLB+AFB about which lesions to biopsy. The adjunct use of Raman spectroscopy as described herein can make the decision process more objective which may result in the identification of additional preneoplastic lesions at sites initially rejected as biopsy candidates. Secondly, as mentioned above, Raman spectroscopy may identify biomolecular changes in both histologically preneoplastic and non preneoplastic lesions that are markers for development into late stage disease.

Application of the technology described herein is not limited to non-invasive diagnosis. In some embodiments, apparatus as described herein may be used during surgery to classify tissues of lesions that become accessible during surgery.

### Example Application

A bronchoscopist performs a bronchoscopy on a patient and uses a range of imaging modalities (for example AFB+WLB) to identify lesions that merit further investigation. The bronchoscopist is using a bronchoscope equipped with Raman spectroscopy apparatus as described herein. The bronchoscopist places the bronchoscope so that the end of the Raman catheter is adjacent to a lesion of interest and operates the Raman spectroscopy apparatus to acquire one or more Raman spectra for tissue in the lesion. The apparatus analyzes the Raman spectrum in real time and attempts to classify the tissue based on the spectrum. The apparatus generates a signal to the bronchoscopist based on the result of the analysis. As a simple example, the apparatus may display a green light if the analysis indicates a classification of ≦MILD and a red light if the analysis indicates a classification of ≧MOD. In some embodiments, the apparatus may indicate a yellow light if the classification cannot be established clearly (as established by posterior probability falling outside of a range determined by suitably chosen cut-off thresholds for example).

The bronchoscopist may elect to take a biopsy in cases where the apparatus indicates a classification of MOD or in cases where the apparatus fails to make a clear classification after two or more attempts. In cases where the apparatus indicates a classification of ≦MILD the bronchoscopist may elect not to take a biopsy unless the bronchoscopist notices some other factor that suggests that a biopsy from that site would be advisable.

Certain implementations of the invention comprise computer processors which execute software instructions which cause the processors to perform a method of the invention. For example, one or more processors in a medical Raman spectrometer system may implement methods as described herein by executing software instructions in a program memory accessible to the processors. The invention may also be provided in the form of a program product. The program product may comprise any non-transitory medium which carries a set of computer-readable signals comprising instructions which, when executed by a data processor, cause the data processor to execute a method of the invention. Program products according to the invention may be in any of a wide variety of forms. The program product may comprise, for example, physical media such as magnetic data storage media including floppy diskettes, hard disk drives, optical data storage media including CD ROMs, DVDs, electronic data storage media including ROMs, flash RAM, or the like. The computer-readable signals on the program product may optionally be compressed or encrypted.

Where a component (e.g. a software module, processor, assembly, device, circuit, etc.) is referred to above, unless otherwise indicated, reference to that component (including a reference to a “means”) should be interpreted as including as equivalents of that component, any component which performs the function of the described component (i.e., that is functionally equivalent), including components which are not structurally equivalent to the disclosed structure which perform the function in the illustrated exemplary embodiments of the invention.

As will be apparent to those skilled in the art in the light of the foregoing disclosure, many alterations and modifications are possible in the practice of this invention without departing from the spirit or scope thereof. Accordingly, the scope of the invention is to be construed in accordance with the substance defined by the following claims.

1. Apparatus for characterization of lung tissues, the apparatus comprising:
a Raman spectrometer configured to generate a Raman spectrum in the relative wavenumber range of 1500 cm⁻¹ to 3400 cm⁻¹;
a Raman spectrum analysis unit configured to characterize tissues on the basis of features in the relative wavenumber range of 1500 cm⁻¹ to 3400 cm⁻¹ of the Raman spectrum; and
a feedback device capable of being driven in response to an output of the Raman spectrum analysis unit to produce a human-perceptible signal indicative of a characterization of the tissues by the Raman spectrum analysis unit.

2. Apparatus according to claim 1 wherein the Raman spectrum analysis unit is configured to process the Raman spectrum to provide smoothed 2^{nd} order derivative spectrum and to characterize tissues on the basis of features in the smoothed 2^{nd} order derivative spectrum.

3. Apparatus according to claim 2 wherein the Raman spectrum analysis unit is configured to generate the smoothed 2^{nd} order derivative spectrum by applying a Savitzky-Golay six point quadratic polynomial to each spectrum.

4. Apparatus according to claim 1 wherein the Raman spectrum analysis unit is configured to process the Raman spectrum by performing a 3-point smoothing operation and to characterize tissues on the basis of features in the 3-point smoothed spectrum.

5. Apparatus according to claim 1 wherein the Raman spectrum analysis unit is configured to base the characterization on first features in a first relative wavenumber range of 1550 cm⁻¹ to 1800 cm⁻¹ and second features in a second relative wavenumber range of 2700 cm⁻¹ to 3100 cm⁻¹.

6. Apparatus according to claim 1 wherein the Raman spectrum analysis unit is configured to analyze the Raman spectrum by computing principal component scores of the spectrum for principal components derived from a training set of Raman spectra comprising components in the relative wavenumber range of 1500 cm⁻¹ to 3400 cm⁻¹ and performing a discriminant analysis according to a discriminant function based on the principal component scores.

7. Apparatus according to claim 6 wherein the discriminant analysis comprises linear discriminant analysis.

8. Apparatus according to claim 6 wherein the Raman spectrum analysis unit comprises a data store and information characterizing the principal components is stored in the data store.

9. Apparatus according to claim 8 wherein the discriminant function is stored in the data store.

10. Apparatus according to claim 1 wherein the Raman spectrum analysis unit comprises a fluorescence background subtraction stage configured to subtract a fluorescence background from the Raman spectrum.

11. Apparatus according to claim 10 wherein the fluorescence background subtraction stage is configured to perform a polynomial fitting routine to estimate a fluorescence background.

12. Apparatus according to claim 10 wherein the Raman spectrum analysis unit comprises a normalization stage following the fluorescence background subtraction stage, the normalization stage configured to normalize the Raman spectrum.

13. Apparatus according to claim 1 wherein the Raman spectrum analysis unit is configured to subtract an ambient background signal from the Raman spectrum.

14. Apparatus according to claim 1 comprising a bronchoscope wherein the Raman spectrometer comprises a light guide insertable into an instrument channel of the bronchoscope to receive light containing the Raman spectrum.

15. Apparatus according to claim 3 wherein the Raman spectrometer analysis unit comprises a normalization stage configured to normalize the Raman spectrum by summing the squared derivative values of each spectrum and then dividing each variable by this sum.

16. Apparatus according to claim 6 wherein the Raman spectrometer analysis unit is configured to characterize the tissues by:
characterizing the tissue in a first category if a posterior probability of a characteristic of the tissue is less than a first threshold;
characterizing the tissue in a second category if the posterior probability of the characteristic of the tissue is greater than a second threshold; and
characterizing the tissue in a third category if the posterior probability of the characteristic of the tissue is between the first and second thresholds.

17. Apparatus according to claim 16 wherein the first threshold represents a cutoff of 0.3±10% and the second threshold represents a cutoff of 0.7±10%.

18. Apparatus according to claim 16 wherein the feedback device produces a human-perceptible signal wherein the signal is:
a first signal if the tissue is in the first category;
a second signal if the tissue is in the second category; and
a third signal if the tissue is in the third category.

19. A method for tissue characterization comprising:
obtaining at least one Raman spectrum of a tissue, the Raman spectrum comprising features in the relative wavenumber range of 1500 cm⁻¹ to 3400 cm⁻¹;
in a programmed spectrum analysis unit comprising a data processor executing software instructions, automatically characterizing tissues at least in part on the basis of features in the 1500 cm⁻¹ to 3400 cm⁻¹ relative wavenumber range of the Raman spectrum; and
controlling a feedback device to produce a human-perceptible signal indicative of the characterization of the tissues.

20. A method according to claim 19 comprising performing a fluorescence background subtraction step to remove a fluorescence background from the Raman spectrum prior to characterizing the tissues.

21. A method according to claim 20 comprising normalizing the Raman spectrum following the fluorescence background subtraction step.

22. A method according to claim 19 wherein the Raman spectrum is processed to provide a smoothed 2^{nd} order derivative spectrum prior to characterizing the tissues and characterizing the tissues is on the basis of features in the smoothed 2^{nd} order derivative spectrum.

23. A method according to claim 22 wherein the smoothed 2^{nd} order derivative spectrum is provided by applying a Savitzky-Golay six point quadratic polynomial to the Raman spectrum.

24. A method according to claim 19 wherein the Raman spectrum is processed by performing a 3-point smoothing operation on the Raman spectrum prior to characterizing the tissues and characterizing the tissues is on the basis of features in the 3-point smoothed spectrum.

25. A method according to claim 19 wherein characterizing the tissues is based on first features in a first relative wavenumber range of 1550 cm⁻¹ to 1800 cm⁻¹ and second features in a second relative wavenumber range of 2700 cm⁻¹ to 3100 cm⁻¹.

26. A method according to claim 19 wherein the Raman spectrum is processed by computing principal component scores of the spectrum for principal components derived from a training set of Raman spectra comprising components in the relative wavenumber range of 1500 cm⁻¹ to 3400 cm⁻¹ and performing a discriminant analysis according to a discriminant function based on the principal component scores.

27. A method according to claim 26 wherein the discriminant analysis comprises linear discriminant analysis.

28. A method according to claim 27 wherein the programmed Raman spectrum analysis unit comprises a data store and information characterizing the principal components is stored in the data store.

29. A method according to claim 28 wherein the discriminant function is stored in the data store.

30. A method according to claim 21 wherein the step of performing a fluorescence background subtraction step comprises performing a polynomial fitting routine to estimate a fluorescence background.

31. A method according to claim 19 comprising the step of subtracting an ambient background signal from the Raman spectrum.

32. A method according to claim 23 comprising the step of normalizing the Raman spectrum by summing the squared derivative values of each spectrum and then dividing each variable by this sum.

33. A method according to claim 26 wherein characterizing the tissues comprises the use of a probability threshold.

34. A method according to claim 26 wherein characterizing the tissues comprises:
characterizing the tissue in a first category if a posterior probability of a characteristic of the tissue is less than a first threshold;
characterizing the tissue in a second category if the posterior probability of the characteristic of the tissue is greater than a second threshold; and
characterizing the tissue in a third category if the posterior probability of the characteristic of the tissue is between the first and second thresholds.

35. A method according to claim 34 wherein the first threshold represents a cutoff of 0.3±10% and the second threshold represents a cutoff of 0.7±10%.

36. A method according to claim 34 wherein controlling the feedback device comprises:
producing a first signal if the tissue is in the first category;
producing a second signal if the tissue is in the second category; and
producing a third signal if the tissue is in the third category.

37. A non-transitory tangible computer-readable medium storing instructions for execution by at least one data-processor that, when executed by the data-processor cause the data processor to execute a method for characterizing tissue comprising the steps of:
receiving at least one Raman spectrum of a tissue the Raman spectrum comprising features in the relative wavenumber range of 1500 cm⁻¹ to 3400 cm⁻¹;
characterizing tissue at least in part on the basis of features in the 1500 cm⁻¹ to 3400 cm⁻¹ relative wavenumber range of the Raman spectrum; and
generating an indication of the characterization of the tissue.

38. The non-transitory tangible computer-readable medium of claim 37 wherein the non-transitory tangible computer-readable medium further stores the at least one Raman spectrum.
